(19) United States
(12) Patent Application Publication
(10) Pub. No.: US 2019/0175784 A1
(43) Pub. Date: Jun. 13, 2019

(54) HYALURONIC ACID GEL WITH HIGH AVERAGE DIAMETER PARTICLE SIZE, A CONSUMER PACKAGE CONTAINING THE GEL AND A USE THEREOF

(71) Applicant: Prollenium Medical Technologies, Inc., Aurora (CA)

(72) Inventors: Timothy Lee, Aurora (CA); Yan Li, Aurora (CA)

(21) Appl. No.: 16/325,754

(22) PCT Filed: Aug. 18, 2017
(86) PCT No.: PCT/CA2017/050985

§ 371 Date: Feb. 15, 2019

Related U.S. Application Data
(60) Provisional application No. 62/376,508, filed on Aug. 18, 2016.

(51) Int. Cl. A61L 27/20; A61L 27/52

(52) U.S. Cl. CPC A61L 27/20; A61L 2430/34; A61L 2400/06; A61L 27/52

(57) ABSTRACT

The dermal filler is of the type having particles of cross-linked hyaluronic acid. At least 10% of the particles have a diameter greater than 800 um; the particles can absorb 5-7 times their weight in water; and the degree of cross-linking is about 7%.

### FIELD OF THE INVENTION

The invention relates to the field of dermal fillers.

### BACKGROUND

It is well known to inject hyaluronic acid under the dermis for cosmetic reasons.

### SUMMARY OF THE INVENTION

Forming one aspect of the invention is an improved dermal filler of the type having particles of cross-linked hyaluronic acid. The improvement comprises: at least 10% of the particles having a diameter greater than 800 um; the particles can absorb 5-7 times their weight in water; and the degree of cross-linking is about 7%.

According to another aspect, about half of the particles can have a diameter above 500 μm.

According to another aspect, the filler can be adapted such that an extrusion force of about 2 lbs is sufficient to force the filler at a rate of 4 ml/hr through a 27 Gauge needle.

A consumer package containing more than 1 ml of the improved filler forms another aspect of the invention.

Use of the improved filler for non-face augmentation procedures forms another aspect of the invention.

### BRIEF DESCRIPTION OF THE DRAWINGS

FIG. 1A is a photograph of a sample of the exemplary filler;

FIG. 1B is the volume-weighted size distribution of the particles of FIG. 1A;

FIG. 2A is a photograph of another sample of the exemplary filler;

FIG. 2B is the volume-weighted size distribution of the particles of FIG. 2A;

FIG. 3A is a photograph of another sample of the exemplary filler;

FIG. 3B is the volume-weighted size distribution of the particles of FIG. 3A;

FIG. 4A is a photograph of another sample of the exemplary filler;

FIG. 4B is the volume-weighted size distribution of the particles of FIG. 4A;

FIG. 5A is a photograph of another sample of the exemplary filler;

FIG. 5B is the volume-weighted size distribution of the particles of FIG. 5A;

FIG. 6A is a photograph of another sample of the exemplary filler;

FIG. 6B is the volume-weighted size distribution of the particles of FIG. 6A;

FIG. 7A is a photograph of another sample of the exemplary filler;

FIG. 7B is the volume-weighted size distribution of the particles of FIG. 7A;

FIG. 8A is a photograph of another sample of the exemplary filler;

FIG. 8B is the volume-weighted size distribution of the particles of FIG. 8A;

FIG. 9A is a photograph of another sample of the exemplary filler;

FIG. 9B is the volume-weighted size distribution of the particles of FIG. 9A;

FIG. 10A is a photograph of another sample of the exemplary filler;

FIG. 10B is the volume-weighted size distribution of the particles of FIG. 10A;

FIG. 11A is a photograph of another sample of the exemplary filler;

FIG. 11B is the volume-weighted size distribution of the particles of FIG. 11A;

FIG. 12A is a photograph of another sample of the exemplary filler;

FIG. 12B is the volume-weighted size distribution of the particles of FIG. 12A.

### DETAILED DESCRIPTION OF THE EXEMPLARY EMBODIMENT

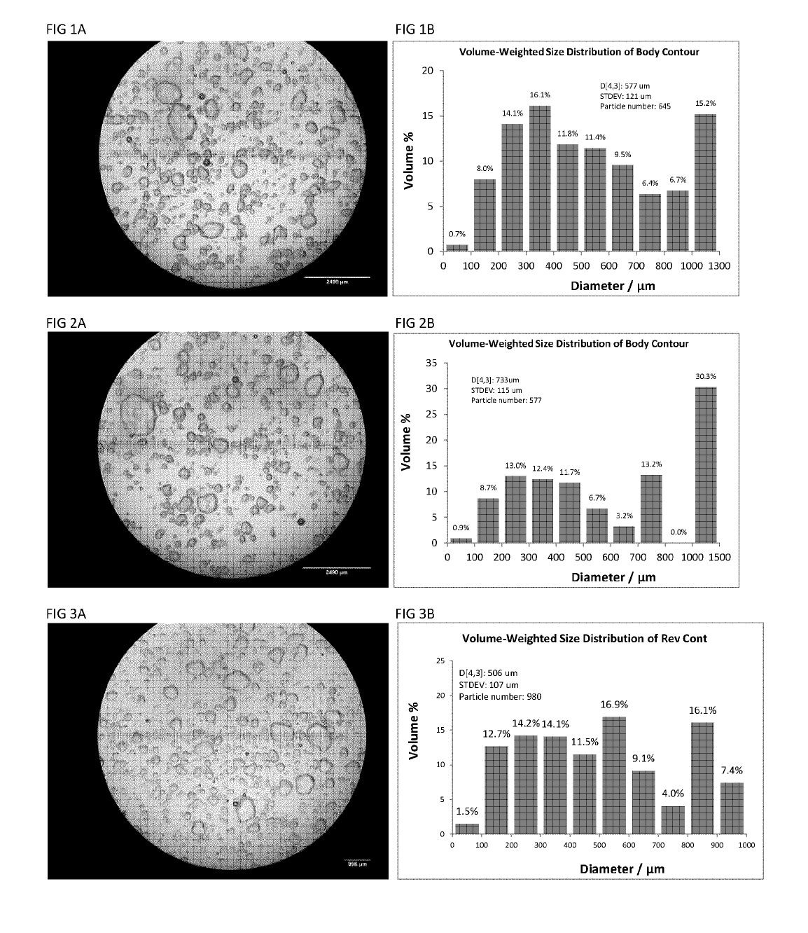

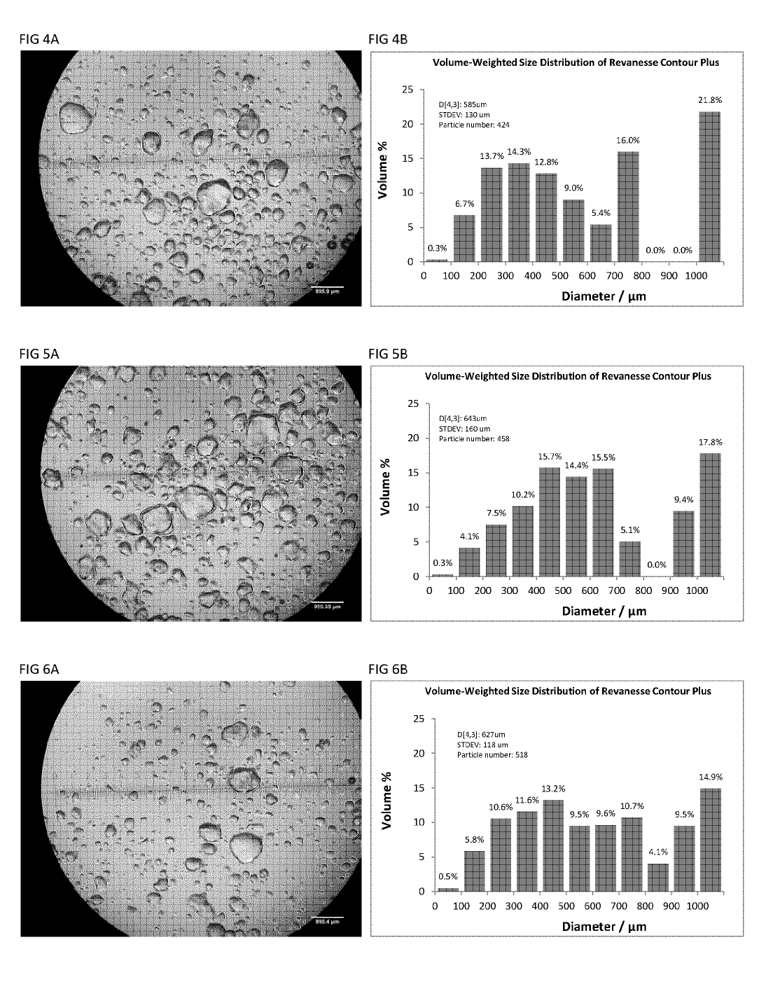

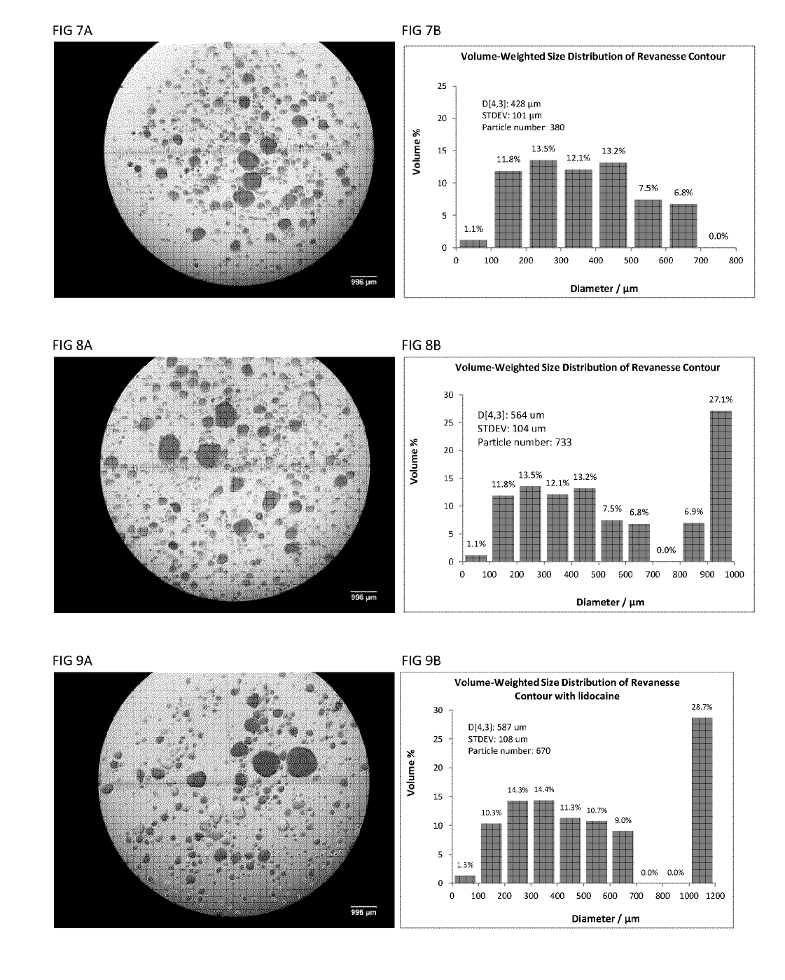

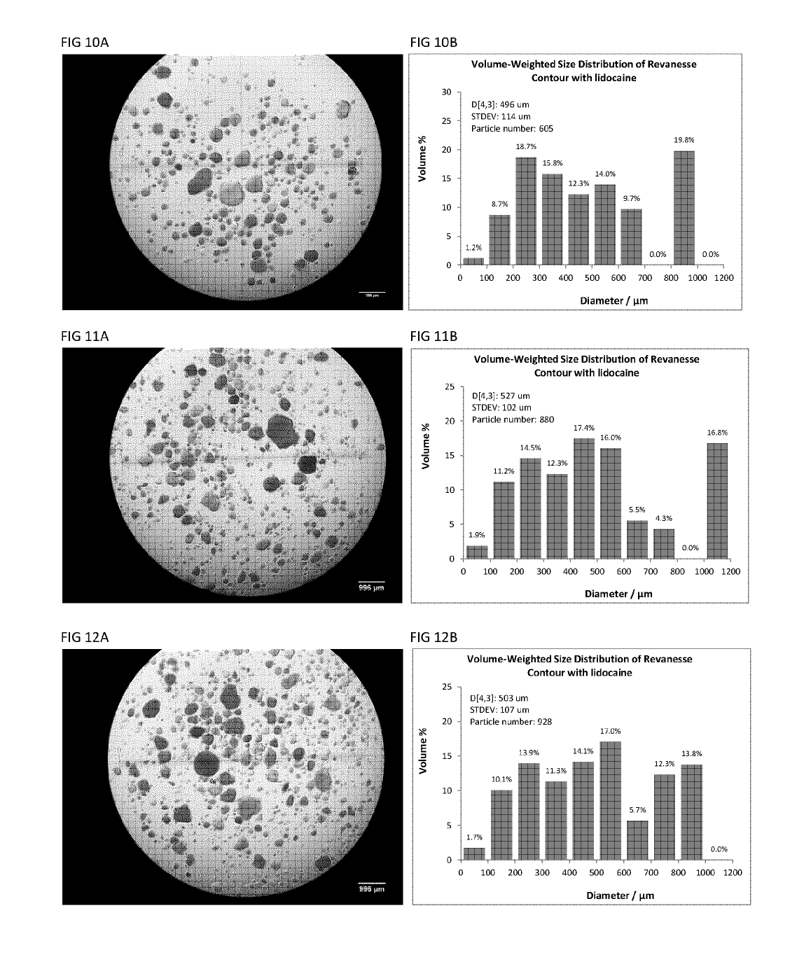

FIGS. 1A-12A show the filler according to the exemplary embodiment.

More particularly, twelve samples of the filler were taken, at random, and photographed under high magnification: these photographs constitute FIGS. 1A-12A.

As will be seen, the filler is characterized by a relatively high average diameter particle size.

The particles in the photographs were counted and measured, such that a volume-weighted size distribution could be produced, as shown by FIGS. 1B-12B.

From these results, the filler can be characterized in that:
at least 10% of the hyaluronic acid particles are greater than 800 um in diameter
about half of the particles in the gel have a particle size above 500 μm.

Further measurements were also taken, from which it was determined that:
the particles can absorb 5-7 times their weight in water
the degree of cross-linking is about 7%
an extrusion force of about 2 lbs is sufficient to force gel at a rate of 4 ml/hr through a 27 Gauge needle.

It has been found useful to inject gel according to the invention into the hips of patients; more particularly, it has been found that the gel has a sufficient low viscosity such that the relatively large volumes needed [above 40 ml] for non-facial [large volume] augmentation can be injected without undue discomfort. 60 ml was injected into the legs of an individual in less than 3 hours without causing any pain or adverse reactions. Accordingly, a consumer package containing more than 1 ml of the gel has been found to be useful.

1. An improved dermal filler of the type having particles of cross-linked hyaluronic acid, the improvement comprising:
at least 10% of the particles having a diameter greater than 800 um
the particles can absorb 5-7 times their weight in water and
the degree of cross-linking is about 7%.

2. The filler according to claim 1, wherein about half of the particles have a diameter above 500 μm.

3. The filler according to claim 1, wherein the filler is adapted such that an extrusion force of about 2 lbs is sufficient to force the filler at a rate of 4 ml/hr through a 27 Gauge needle.

4. A consumer package containing more than 1 ml of the improved filler.

5. Use of the improved filler for non-face augmentation procedures.
